(19) United States
(12) Patent Application Publication
(10) Pub. No.: US 2019/0224489 A1
(43) Pub. Date: Jul. 25, 2019

(54) Alzheimer's Treatment Using Ultra-Low Magnetic Field Oscillations

(71) Applicants: Harold Benjamin Liemohn, Puyallup, WA (US); Harrison Robert Willard, Bothell, WA (US)

(72) Inventors: Harold Benjamin Liemohn, Puyallup, WA (US); Harrison Robert Willard, Bothell, WA (US)

(73) Assignee: Laurence Anthony Deas, Puyallup, WA (US)

(21) Appl. No.: 15/839,371

(22) Filed: Jan. 22, 2018

(51) Int. Cl. A61N 2/00; A61B 5/00; A61N 2/02

(52) U.S. Cl. CPC A61N 2/006; A61B 5/6803; A61N 1/36014; A61B 5/4088; A61N 2/02

(57) ABSTRACT

We propose to patent the treatment of Alzheimer's disease and other neurodegenerative diseases in the central nervous system using the external application of ultra-low-intensity oscillating magnetic fields in the gamma frequency band that will stimulate neuron currents and activate the natural healing processes of microglia. Observed magnetoencephalographic emissions are thought to be generated by neuron-cluster current loops producing equivalent current dipoles (ECDs). Our analysis of these ECDs concludes that adequate neuron stimulation will require a field threshold above a pico-Tesla. Some experimental testing will be required to ascertain the threshold for effective microglial activation. Our invention is human headgear containing a set of orthogonal current coils for magnetic stimulation of neurons to activate brain microglia. Other coil configurations may be useful for ganglia activation elsewhere in the body.

### BACKGROUND OF INVENTION

We propose to patent the treatment of Alzheimer's disease and other neurodegenerative diseases using the external application of ultra-low-intensity oscillating magnetic fields between 10⁻¹² and 10⁻⁹ Tesla in the alpha through gamma frequency bands (5 to 100 Hz). Our invention is human headgear containing a set of orthogonal current coils for magnetic stimulation of neurons to activate the natural healing processes of brain microglia. Other coil configurations may be useful for ganglia activation elsewhere in the body.

The success of a flickering light experiment to stimulate sufficient neuron current to activate microglia destruction of plaque in the visual cortex of mice motivated our exploration of stimulation by magnetic fields. For human neuron stimulation, the field emissions measured with magnetoencephalography (MEG) observations of placid subjects provide an initial baseline. For cortical activity in the gamma band, these MEG fields are only about 10 femto-Tesla (10⁻¹⁴T). Our conclusion is that very low level oscillating fields will be required to stimulate the neuron currents necessary for microglial activation to destruct and eliminate the amyloid plaque that is believed to be largely responsible for the Alzheimer's disease.

However, the ambient currents that generate these MEG emissions emanate from neuron clusters with dipole-field characteristics that imply the source fields are significantly greater than observed levels. Our analysis of these dipoles predicts adequate neuron stimulation for treatment will require magnetic fields somewhat above 10⁻¹² Tesla. Determination of the lowest field level that will activate microglial healing requires exposing mice infected with Alzheimer's and observing their improvements in behavioral responses. Because chronic or hyper-activation of microglia can generate damaging cytotoxins, it will also be desirable to assess the breadth of safe ranges for their treatment parameters.

Finding the most helpful time intervals and field intensities for the treatment of human patients with Alzheimer's disease will require an orderly series of test exposures. Since the applied magnetic fields and induced currents are so small, we believe there is no reason for concern about residual harm or adverse side effects. Our configuration for application of orthogonal fields that would stimulate all neuron orientations in the brain is illustrated in FIG. 1. For human treatment, the optimum exposure regimen would be determined by measuring significant improvements in the memory-recall capabilities and conversation patterns of patients through standard testing techniques.

This magnetic field application should be considered a treatment for temporary relief from the symptoms rather than a cure for Alzheimer's. Clearly it does not deter the fundamental process that generates the amyloid plaque and other detritus believed to produce human diseases in the central nervous system. If the application of these magnetic fields can provide some measure of relief for the symptoms of Alzheimer's disease, it would give many seniors a more benevolent and meaningful end of life. Possible suppression of symptoms for other neurodegenerative diseases would be a bonus.

### REFERENCES CITED

1 U.S. Pat. No. 9,456,784 October 2016 Heleka
2 U.S. Pat. No. 9,233,257 January 2016 Zabara
3 U.S. Pat. No. 8,868,177 B2 October 2014 Simon, et al
4 U.S. Pat. No. 7,744,523 B2 June 2010 Epstein
5 U.S. Pat. No. 5,766,124 6/1998 Poison
6 Tsai, L.-H., et al, Brain Wave Stimulation, Nature, Dec. 7, 2016.
7 Jackson, J. D., Classical Electrodynamics, 1962, p 177.
8 Dheen, S. T., Kaur, C. and Ling, E. A., Microglial activation and its implications in the brain diseases, Current Medical Chemistry, 2007; 14 (11): 1189-97.
9 Nakamura, Y., Regulating Factors for Microglial Activation, Biological and Pharmaceutical Bulletin, August 2002; 25(8), 945-953.
10 Wikipedia, Microglia, Internet Free Encyclopedia.
11 Gehrmann, J., Matsumoto, Y., and Kreutzberg, G. W., (1995) Microglia: Intrinsic immuneffector cell of the brain, Brain Research Reviews, 20 (3): 269-87.
12 Palva, J. M., Palva, S., and Kaila, K., Phase Synchrony among Neuronal Oscillations in the Human Cortex, The Journal of Neuroscience, Apr. 13, 2005; 25(15): 3962-3972, FIGS. 1A and 9A.
13 Cohen D. (1972), Magnetoencephalography: detection of the brain's electrical activity with a superconducting magnetometer, Science175: 664-66.
14 Wikipedia, Magnetoencephalography, Internet Free Encyclopedia.
15 Jackson, J. D., Classical Electrodynamics, 1962, p 225.
16 Rush, S., Abildskov, J. A., and McFee, R., Resistivity of Body Tissues at Low Frequencies, Circulation Research, Vol XII, January, 1963, 40-50.
17 Schimpf, P. H., Ramon, C., and Haueisen, J., Dipole Models for the EEG and MEG, IEEE Transactions on Biomedical Engineering, v 49, May, 2002, 409-418.
18 Perin, R. Telefont, M., and Markram, H., Computing the size and number of neuronal clusters in local circuits, Frontiers in Neuronalanatomy, 2013; 7:1.
19 Jackson, J. D., Classical Electrodynamics, 1962, p 143.
20 Sears, F. W., and Zemansky, M. W., University Physics, 2^{nd} Ed., 1955, p.595.
21 Wikipedia, Helmholtz Coil, Internet Free Encyclopedia.

### BRIEF SUMMARY OF INVENTION

We propose a treatment for Alzheimer's disease and other neurodegenerative diseases using the external application of ultra-low-intensity oscillating magnetic fields between 10⁻¹² and 10⁻⁹ Tesla in the alpha through gamma frequency bands (5 to 100 Hz). Our invention is human headgear containing a set of orthogonal current coils for magnetic stimulation of neurons to activate the natural healing processes of brain microglia. Other coil configurations may be useful for ganglia activation elsewhere in the body.

### DESCRIPTION OF DRAWINGS

FIG. 1. Sketch of our field-coil configuration for human treatment of Alzheimer's disease. On the left is a continuous-wire plan view, and on the right is a 3-dimensional sketch of the orthogonal array mounted in place. Opposing side coils in the Helmholtz configuration have current arrows in the same direction.

FIG. 2. Block diagram of the current oscillator for the field coils including frequency and current level controls. Because very small currents are needed to produce the applied magnetic fields, a small integrated circuit driven by AA batteries appears adequate to generate the required oscillator current.

### DETAILED DESCRIPTION OF INVENTION

For many decades, medical research has utilized electric shock therapy and magnetic stimulation of brain tissue to improve performance and behavior of patients with a variety of abnormalities. These treatments have consisted of large-current pulses and/or high-strength magnetic fields to stimulate target nerve bundles in the central nervous system in order to achieve desired effects and responses in the patient.

Many different treatment methods utilizing some form of magnetic field stimulation have been developed and patented during the last few decades. For example, a recent patent, U.S. Pat. No. 9,456,784 by Helekar¹, described an apparatus for exposing the head of a patient to rotating transcranial magnetic stimulation (rTMS). The device utilizes permanent magnets with maximum pole strength of 1.48 Tesla rotating at a repetition rate of 0.1 to 2 Hz to induce weak electric currents in the brain. The level of field strength exposure from this device is many orders of magnitude larger than what is proposed herein.

U.S. Pat. No. 9,233,257 by Zabara² describes methods for exposing patients to electromagnetic radiation fields utilizing a wide range of emission parameters. The radiation source is confined to the low and radio frequency bands, well above the gamma frequency band that we propose to use. Pulses of 0.3 seconds or less apply radiation currents of 0.0001 to 1 ampere and magnetic fields between 0.5 and 10 Tesla to selected target areas in the brain. Such field levels also far exceed those proposed here.

U.S. Pat. No. 8,868,177 by Simon, et al,³ proposes using a large external magnetic field to drive a current through its conducting core that passes through a conducting gel into the patient at its target—the vagus nerve. The applied magnetic field does not penetrate the patient directly, but its strength is many orders of magnitude higher than proposed here. It serves only to generate voltages across and current through the gel pads.

U.S. Pat. No. 7,744,523 by Epstein⁴ describes a repetitive transcranial magnetic stimulation (rTMS) device that claims to induce voltages of 0.09 volts in the brain's nerve tissue. Such an impulse requires inducing electric fields in excess of 1 volt/cm, much greater than the electric fields we calculate to drive neuron currents.

U.S. Pat. No. 5,766,124 by Polson⁵ describes several electrical devices that apply magnetic fields to a patient's head to treat Alzheimer's dementia. But the rate of change of their fields is typically 20,000 Tesla/second. At 40Hz this rate produces a 70 Tesla peak field strength, about 10⁺¹² times our proposed levels.

All of these recent patents as well as earlier ones apply magnetic field strengths that are many orders of magnitude above the levels we require for this patent. Our research suggests that applying ultra-low magnetic field strengths in the gamma band will adequately activate the natural defense mechanisms in the central nervous system to stimulate healing of neurodegenerative diseases.

Recent research at MIT by Dr. Li-Huei Tsai and her colleagues⁶ exposed mice with the Alzheimer's disease to 40 Hz flickering light and found that it stimulated neuron currents only in the optical cortex. They report that these currents activated microglia to attack and break up local amyloid-protein plaque structures that are believed to be responsible for the symptoms of Alzheimer's. Mice treated with the light stimulation displayed more responsive behavior and were able to better recall maze structure pathways.

Such results suggest that appropriate neuron excitation by other means would also generate the microglial activity that promotes natural healing processes to cleanse the plaques in the brain allowing Alzheimer's patients to regain more normal behavioral responses. One well-known process that generates currents in conducting media is the application of time-varying magnetic fields. Specifically, Faraday's Law in Maxwell's electromagnetic equations⁷ quantitatively describes how oscillating magnetic fields in conductors generate electrical fields that can drive electrical currents. Consequently, we believe their application in the appropriate human frequency band (5-100 Hz) and field strength will cause similar stimulation of local neuron currents in large regions of the brain. Furthermore, stimulation of ganglia may allow activation of microglia and break up the plaque elsewhere in human anatomy that causes a variety of maladies of the central nervous system.

According to a research review on microglial activation by Dheen, et a1,⁸ these resident immune-defense cells are the principal responders in the central nervous system that can attack and remove a variety of cell structures responsible for a neurodegenerative diseases such as Parkinson's, multiple sclerosis, and HIV-dementia as well as Alzheimer's. In this respect they perform a task similar to white blood cells in our circulation system. However, overstimulation or chronic activation of microglia may also cause detrimental neuronal damage through the release of cytotoxic molecules. An earlier review by Nakamura⁹ reports several diseases where hyperactivation of microglia may be responsible for neuronal degeneration. Thus it will be necessary to ascertain the minimum magnetic field threshold that produces effective microglial activation. In addition, it will be important to monitor and assess the response of patients to any treatment regimen.

However, microglia account for 10-15 percent of all brain cells,¹⁰ so their activation offers a truly dominant mechanism for scavenging plaques, damaged neurons, synapses and infectious agents.¹¹ Experimental procedures for determining this activation threshold are described below. Furthermore, the ultra-low-level magnetic fields used here for effective stimulation are so small that they are very unlikely to generate an overreaction. And any concerns about chronic activation will be avoided with proper medical supervision.

The minimum field strength necessary to generate neuron currents sufficient to activate the microglia can be estimated from measurements of magnetic fields emitted by brain-wave activity. According to magnetoencephalographic (MEG) measurements,^{1213,14} normal cortical emissions in the gamma band (25-100 Hz) are on the order of only 10 femto-Tesla (10⁻¹⁴ T) and up to 1 pico-Tesla (10⁻¹² T) for alpha rhythms (7.5-12.5 Hz).

Because human tissue has electrical conduction properties, it is relevant to determine if these observed MEG field emissions are dissipated by local eddy currents in surrounding tissue as they propagate from their cortical sources to the superconducting sensors^{13′14} outside the body. The technical measure of such signal damping is characterized by the skin depth δ, the thickness of the propagation medium wherein the initial wave amplitude decreases exponentially by 1/e=0.369. Its value depends on the resistivity or conductivity of the medium and is defined (in mks units) by the expression¹⁵

δ=(2/μωσ)^{1/2}=(ρ/μπf)^{1/2},

where μ is the permeability of the medium, ω is the angular frequency, σ is the conductivity of the medium, ρ is resistivity of the medium, and f is signal frequency.

The resistivity, p of various types of human tissue has been measured by five independent research groups at frequencies of interest for this application and their values are summarized in Rush, et al.¹⁶ Relevant values for the head ranged from ˜100 Ohm-centimeters for blood and ˜230 Ohm-cm for muscle, up to 1800 Ohm-cm for skeletal and 2000+ Ohm-cm for fat tissue. To illustrate the skin depth of wave propagation in human tissue at gamma frequencies, we will use a low-end mean value for human tissue of 200 Ohm-cm, or (in mks units) 2 Ohm-meters. Since the human anatomy is not magnetic we use the free space permeability, μₒ=4π×10⁻⁷ Tesla-meter/Ampere. And lastly, we will use a frequency of 40 Hz after the success of the flickering light experiment by Dr. Tsai, et al⁶. These estimated values yield a skin depth, δ of 113 meters. Thus, it is reasonable to conclude that there is no attenuation of gamma frequency signals for the entire range of measured resistive values in the magnetic field pathways of human tissues. Therefore, the externally measured MEG signals are the actual values of the magnetic fields generated by neuron currents. And, externally applied fields in this frequency band undergo no losses entering human anatomy.

Although magnetic signals are not attenuated by eddy currents in head tissue, the MEG fields are believed to emanate from “point-source” neuron clusters in the central nervous system producing equivalent current dipoles (ECDs).^{14.17} Such dipole fields diminish with distance, r, away from their sources as 1/r³, so the MEG field (10⁻¹⁴ Tesla) that is measured with ultra sensitive SQUIDs^{13,17} (Superconducting QUantum Interference Devices) is much less than the neuron source emissions.

Therefore, to stimulate the neuron currents above normal levels, the applied field must be somewhat stronger than the ECD source field of the active clusters. In order to determine this source field, a simple cluster-field model will suffice. Quantitative characteristics for our model were selected from average values found in Perin, et al,¹⁸ for the size and number of neuronal clusters and networks. The human brain has billions of nerve cells that interact with close neighbors forming clusters consisting of only a few dozen neurons. A few thousand of these clusters provide the structure for each local network that responds to external stimuli. And within the brain there is a large collection of these operational networks.

We assume the observed MEG field is generated by the ECDs in n active clusters in the collection of networks throughout the brain. The ECD produced by each cluster will be represented by a dipole moment, mₖ=tₖaₖ, where ιₖ (iota) is the cluster dipole current and aₖ is the cluster area in meters. For estimating the total field, it will be assumed that all of these clusters are arbitrarily located an average distance d=7.5 centimeters from the SQUID sensor array, which is normally located close to the skull in a helmet. With this approximation the dipole moment of the active network collection in the brain may be represented by the summation

$$m = {\sum }_{k}^{n} {m}_{k}$$

Such a configuration model is not intended to be definitive, but offers adequate guidance for estimating the stimulation threshold.

To evaluate the order of magnitude of m, we will use the well-known features of magnetic dipoles. Because the sensors are located in the distant field (d>>>a^{1/2}), the radial and angular components of the collective dipole field (in mks units) are given by the simple expressions,¹⁹

Bᵣ=(2μₒm/4πr³) cos θ

and

B_{θ}=(μₒm/4πr³) sin θ,

where r is the radial distance from the center of the dipole and θ is the angle above or below the source-loop plane. To accommodate the random orientations of the dipole loops, assume θ is 45°, so sin θ=cos θ=2^{-1/2}. Then the observed scalar field for the collection of n clusters is

B_{obs}=(Bᵣ²+B_{θ}²)^{1/2}=(5/2)^{1/2}(μₒm/4πd³).

Since B_{obs} is 10⁻¹⁴ Tesla, this relation may be solved for the magnetic moment, m=2.7×10⁻¹¹ Ampere-meters². It is noteworthy that its value is only dependent on the sensor distance d and changes by only a factor of 2 for a two centimeter increase in d.

This approximate value for the magnetic moment of the entire brain will be used to estimate the effective field threshold for an individual cluster using the well known Biot-Savart law²⁰ for the field, Bₗ, at the center of a single loop, l, of current,

Bₗ=μₒιₗ/2ρₗ,

where ιₗ (iota) is the loop current and ρₗ (rho) is the loop radius. Employing the characteristic average values from Perin, et al,¹⁸ it will be assumed that 2000 clusters are contained in the local network. Then in the collection of active networks there are 2000 υ clusters where υ (nu) is the number of contributing networks. Thus the loop current, ιₖ, for an average single cluster is

ιₖ=m/2000 υaₖ.

Inserting this value in the Biot-Savart relation, we arrive at the expression for an average dipole field at the center of the kth cluster loop,

Bₖ=μₒ(m/2000 υaₖ)/2ρₖ=10⁻¹⁰ m/υρₖ³.

Because the distance between adjacent neurons in the cluster is about 100 to 150 micrometers, we assume the cluster radius Σₖ for a few dozen neurons must be at least 300 micrometers. And for this evaluation, the number υ of contributing networks is arbitrarily assumed to be 100. For these parameters we arrive at the cluster ECD field value, Bₖ=10⁻¹² Tesla, for our threshold estimate.

In order to determine the optimum magnetic field strength that achieves effective activation of the microglial breakup of plaque, a range of intensities from 10⁻¹² to 10⁻⁹ Tesla will be investigated experimentally to determine the desired field exposure. But it should be emphasized that chronic over activation must be avoided.⁹

We want to emphasize that these exceptionally low magnetic field strengths are unique to the claims of this patent.

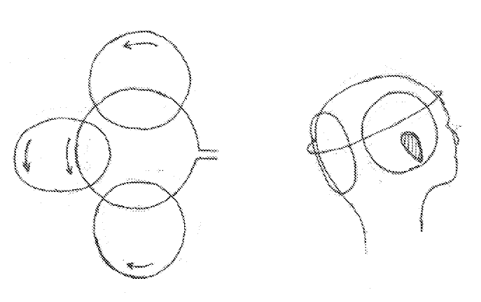

For medical applications the magnetic field can be created using simple circular coils with dimensions that will cover the area to be treated. In most cases a geometric array of orthogonal coils will be needed to assure stimulation of current for all possible orientations of the neurons. An orthogonal coil configuration for treatment of Alzheimer's patients is illustrated in FIG. 1 where the top and back coils operate alone and the side coils (over the ears) are a matching pair with current running in the same direction.

Since these treatment fields will be generated by external circular coils, their magnetic fields at axial distance X along its center-line may also be derived from a more generalized formulation of the Biot-Savart law²¹, which has been rewritten to explicitly illustrate its spatial variance,

B(X)=(μₒNi/R)/2(1+X²/R²)^{3/2}=(μₒNi/R)C(X),

where Ni is the total coil turns times the current, and R is the coil radius in meters. The spatial coefficient, C(X), decreases almost logarithmically with axial distance as shown in the following table:

| | | | | | | | |
|---|---|---|---|---|---|---|---|
| X = | 0 | R/2 | R | 3R/2 | 2R | 5R/2 | 3R |
| C = | 0.50 | 0.36 | 0.18 | 0.096 | 0.045 | 0.026 | 0.016 |

The fall off in C illustrates the need to apply the external field source as close to the target area as possible. Furthermore, the near field of a single coil diverges, so neuron clusters off the coil axis will receive substantially less field strength.

Some treatment locations will allow coil positions on opposite sides of the target area in the Helmholtz configuration, wherein identical solenoid electromagnets are on the same axis and separated by a coil radius. Such an array has the important characteristic of producing a virtually uniform magnetic field fall off between the coils. Its value at the axial midpoint, R/2, is simply²¹

Bₘₚ(R/2)=(8/5^{3/2})(μₒNi/R),

twice the field level of the single coil above. In many applications this approximate uniformity across the entire diameter between the coils provides a desirable feature, especially for treatment of larger areas. Such coil pairs separated by more than R also have a spatial coefficient of 2 C at their axial-field midpoint. However, more separation causes moderate divergence off the coil axis resulting in uniform-field degradation.

Combinations of coil diameters and field strengths may provide additional beneficial focusing of field energy required for specific applications. These situations may necessitate exact analytical solutions for the off-axis field of circular coils requiring advanced mathematics using complete elliptic integrals. This accuracy is not needed for the near-field applications proposed here.

These magnetic field expressions may be solved for the oscillator current amplitude, l, required to provide the desired magnetic field strength. For the amplitude along the single coil axis at X=R, the current-turn solution is

NI=(2^{5/2}R/μₒ)B(R)

As before, assume R=d =7.5 centimeters. Then, to produce the field amplitude, B (7.5 cm)=1×10⁻¹² Tesla, will require a minimal coil-turn-current amplitude NI of 0.34 micro-Ampere-turns.

In order for the applied field energy to be concentrated at the desired operating frequency, the current profile of the driving oscillator should be approximately sinusoidal,

i=l sin (2πft),

where f is the selected operating frequency in the gamma band and t is time.

At these low intensities and frequencies, the inductance of the coil circuit may be neglected since a single loop of wire (heavy-gauge to hold its shape) would be adequate. However, an efficient oscillator design requires many more coil turns to provide optimum impedance matching characteristics.

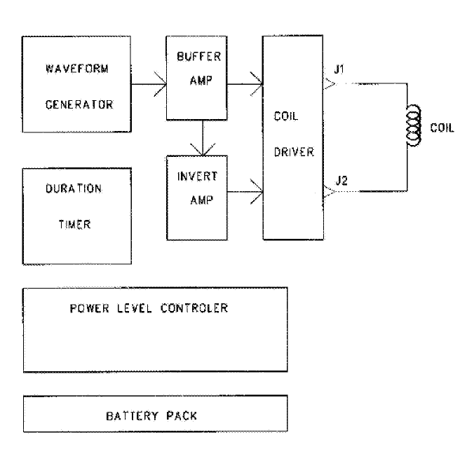

The current source will be a standard electrical oscillator circuit with frequencies in the gamma band, and a range of current amplitudes, I, to determine an appropriate magnetic treatment threshold. A block diagram for such a circuit is shown in FIG. 2. Some design considerations will be illustrated with parameter estimates for the high-end treatment field of 10⁻⁹ Tesla that requires 340 micro-Ampere-turns. In order to optimize operation of the oscillator, the coils will need about 20 turns of very light-weight AWG 29 wire constrained by a circular ring. For a ring diameter of 15 centimeters the orthogonal set of four coils (shown in FIG. 1) will require124 feet of wire. When connected in series, the total impedance of the circuit will be about 10 Ohms (2.5 Ohms per coil). Since the current requirement for this field intensity is about 17 micro-Amperes, the oscillator circuit must provide only 0.17 milli-Volts, easily achieved with a pocket-sized power supply.

With modern CMOS technology, such as a Microchip, Inc.16-bit microcomputer, the oscillator would require only 5 Volts and draw 3 milli-Amperes, so 4 AA batteries would be sufficient. This chip can provide a peak voltage of 2 Volts for frequencies in the gamma band with a sine wave approximation having <5% distortion. Furthermore, it can monitor the battery charge state and monitor treatment time with an automatic time-out shutdown. In mass production we estimate the oscillator parts will cost less than $25. And for daily half-hour treatments the battery pack should last for several months. Of course, final design details for the driver voltage, current amplitude, and frequency will be determined by the coil-design characteristics and the efficacy of the treatment regimen. In any case, for most applications the device will be portable, inexpensive, and easy to operate.

We recommend testing the potency of this treatment by exposing mice infected with Alzheimer's to a range of magnetic field strengths and frequency values. Their cage would need to be non-conducting and non-magnetic so the applied magnetic field would be unaffected. In order to assure thorough stimulation of all neuron orientations in the subjects, the field coils would be arrayed orthogonally outside the cage in a Helmholtz configuration. Based again on the success of the flickering light experiment by Dr. Tsai, et al⁶, we propose to use an initial 40 Hz oscillation frequency for the field-current generator. In order to determine the minimum signals required to achieve acceptable activation, a range of gamma frequencies and applied field strengths will be explored. The efficacy of such a treatment and the magnetic field threshold would be verified by observing changes in the activity levels of the mice and their ability to recall maze pathways in the cage. Because chronic- or hyper-activation of microglia can generate damaging cytotoxins, it would also be desirable to experimentally determine an upper field limit for their treatment regimen.

Response of human patients with Alzheimer's disease to this methodology necessitates a test program that incrementally increases their exposure times and magnetic fields. Because the currents and magnetic fields are so small, we believe there is no concern about residual harm or adverse side effects, but any abnormal responses must be checked. As noted above our configuration for application of orthogonal fields that would stimulate all neuron orientations in the brain is illustrated in FIG. 1. The minimum exposure threshold would be readily ascertained through observations of discernible improvements in the memory-recall capabilities and conversation patterns of patients.

Furthermore, this magnetic field application should be considered a treatment o temporary healing rather than a cure for Alzheimer's. Clearly it does not affect the fundamental process that generates the amyloid plaque and other detritus believed to produce human diseases. But we believe it has the potential to provide temporary to semi-permanent amelioration of the disease symptoms. Medical professionals will need to establish the treatment regimen for each individual based upon their assessment of the patient's physical condition and mental capacity. We anticipate treatments lasting a few minutes to at most one hour and occurring anywhere from daily to monthly. Since the device we envision is extremely simple to build and operate, we believe it would be reasonable to create an in-home treatment schedule.

Furthermore, the low power levels needed to drive the current allow a simple battery driven oscillator using multi-turn coils to generate the fields, so its cost should be very modest. For Alzheimer's treatment, we suggest using a simple array holder (such as a baseball cap) for the coil carrier with an external power supply small enough to fit in a pocket. Treatment for other brain diseases may require different coil arrangements and minor oscillator modifications.

Assuming this treatment is as effective as our analysis would suggest, it seems reasonable that other nerve centers in the body might also benefit from magnetic field stimulation. For example, locations in the torso, where plaque is generated and causes deterioration of human function, might benefit from Helmholtz coil treatments that could stimulate neuron ganglia to activate the local microglial healing process. And early detection of diseases linked to other parts of the nervous system may permit these treatments to significantly delay further development or possibly eradicate the symptoms. To achieve these goals, conceivably the whole body might be exposed to much larger coil configurations.

1. Our invention consists of a set of orthogonal current coils surrounding the human brain that apply magnetic field oscillations driven by a small battery-powered oscillator.

2. Our analysis concludes external application of ultra-low intensity oscillating magnetic fields between pico-Tesla (10⁻¹² T) and nano-Tesla (10⁻⁹ T) or more in the alpha to gamma frequency band (5-100 Hz) are adequate to stimulate neuron currents in the human brain.

3. Neuron current stimulation by such fields will produce microglial activation that attacks and eliminates the cell structures that cause brain diseases in the central nervous system.

4. Removal of these deleterious cell structures will suppress the symptoms of central nervous system diseases such as Alzheimer's, Parkinson's, multiple sclerosis, and HIV-dementia.

5. The treatment threshold will be resolved experimentally by exposing mice and human patients with Alzheimer's to increasing magnetic field amplitudes and observing suppression of their disease symptoms.

6. For Alzheimer's treatment these magnetic fields shall be applied with an orthogonal array of circular coils in close proximity to the patient (FIG. 1).

7. The final design of the power supply (FIG. 2) that drives the coil currents will be determined by the threshold for effective treatment levels in human patients.

8. The opportunity to explore the application of such magnetic field stimulation to promote localized treatments of neurological diseases in other human organs.

9. The opportunity to explore the application of such magnetic field stimulation to the whole body as a prophylactic for suppression of early symptoms of disease and maintenance of general human health.
